(19) United States
(12) Patent Application Publication
(10) Pub. No.: US 2023/0320610 A1
(43) Pub. Date: Oct. 12, 2023

(54) METHOD AND SYSTEM FOR ESTIMATING BRAIN TISSUE DAMAGE WITHIN WHITE MATTER TRACTS FROM A QUANTITATIVE MAP

(71) Applicants: Siemens Healthcare GmbH, Erlangen (DE); Centre Hospitalier Universitaire Vaudois, Lausanne (CH)

(72) Inventors: Veronica Ravano, Pully (CH); Gian Franco Piredda, Lausanne (CH); Tom Hilbert, Lausanne (CH); Tobias Kober, Lausanne (CH); Jonas Richiardi, Geneve (CH)

(21) Appl. No.: 17/716,604

(22) Filed: Apr. 8, 2022

(51) Int. Cl. A61B 5/055; A61B 5/00; G06T 7/00; G01R 33/48

(52) U.S. Cl. CPC A61B 5/055; A61B 5/0042; A61B 5/4064; A61B 5/743; G06T 7/0014; G01R 33/4806; G06T 2207/30016

(57) ABSTRACT

A system and a method for mapping brain tissue damage from quantitative imaging data. The method is implemented by acquiring a quantitative map of a brain tissue parameter of said brain; acquiring a tractography map for said brain; superimposing a first map based on the quantitative map onto a second map based on the tractography map. Metrics are extracted from the superimposition that reflect a distribution of tract-specific quantitative values of the brain tissue parameter and the metrics of the brain are displayed.

### FIELD AND BACKGROUND OF THE INVENTION

The present disclosure is directed, in general, to imaging techniques for imaging biological objects, such as tissues, using for instance Magnetic Resonance Imaging (MRI). More specifically, the present invention is directed to methods and systems for estimating brain tissue damage within white matter tracts from a quantitative map, notably from quantitative MRI (qMRI).

For some neurological pathologies, it has been shown that the amount of focal tissue damage (e.g., lesion, tumor) seen in MRI does not correlate with clinical scores reflecting the patient's well-being. For example, for the clinical examination of multiple sclerosis (MS) patients, radiologists typically evaluate the number of lesions that are visible in the MRI scan. However, this radiological metric (“lesion count”) correlates poorly with a patient's disability level as given by standard clinical metrics such as the “Expanded Disability Status Scale” used in MS. This discrepancy between radiological findings and symptoms is well-known and referred to as the ‘clinico-radiological paradox’ of Multiple Sclerosis [1]. This phenomenon is however also observed in other brain diseases.

To fill this gap, other, more informative measurements derived from diagnostic images (or ‘imaging biomarkers’) can be extracted from MRI scans. For example, diffusion imaging (i.e., probing the directionality of water molecules in tissue through MRI) can be used to derive fiber tracts, i.e., determining the route of bundles of axons within the brain. This allows to see which brain regions are connected, and where the connecting fiber tracts run. This so-called “connectome” view on the brain has shown great potential for characterizing neurological disorders in a more comprehensive manner. Knowing these pathways, focal tissue damage can be situated with respect to the fiber tracts, and hence the patient's symptoms correlated to the function of the brain regions which are connected by the affected fibers rather than just correlating a simple count or similar. To obtain these brain pathway maps (“connectomes”) through techniques called “fiber tracking,” advanced MRI diffusion data have to be available, which is rarely the case in routine clinical examinations.

In the past years, various imaging biomarkers have been investigated to improve the correlation between patient disability and imaging biomarkers. For instance, different studies were conducted to investigate focal damage locations specifically in different white matter (WM) tracts. In 1998, a study on 39 MS patients showed that lesion load on the manually delineated cortical spinal tract correlated better with EDSS than total lesion load [2]. In another study, the authors used the time before the patient requires bilateral support to walk as a disability metric to be correlated with the lesion load in major motor and associative tracts. A significant correlation was found between disability and voxel-wise lesion probability in the corticospinal tract, the superior longitudinal fasciculus and the right inferior fronto-occipital fasciculus [3].

Other methods are based on qMRI. An advantage of qMRI is that it measures absolute physical parameters of the tissue, resulting thus in better comparability between longitudinal scans of the same patients, between multi-site acquisitions or between different cohorts of subjects (e.g., healthy vs. pathological patients) in comparison to conventional “weighted” imaging. As qMRI provides comparable tissue parameters independent from hardware and other spurious effects, it enables the creation of “normative atlases”, i.e., brain maps of tissue parameters which define a range of normal values seen in healthy tissue. Having such an atlas, an individual patient dataset can be checked against it, resulting in a “deviation map” which identifies brain regions where the patient's tissue characteristics differ from what is expected in healthy tissue. For instance, it enables to quantify the extent of diffuse tissue damage (e.g., inflammation, myelin degradation, axonal loss, among others) in normal-appearing tissue, and can thus improve disease characterization. By allowing for such a single-patient assessment, already small changes can be detected, potentially improving diagnosis and follow-up assessments by correlating parameter variations with the undergoing microstructural changes [4]. Another work using qMRI for MS assessment showed that evaluating T1 relaxation time in normal-appearing tissue was a predictor of disease progression longitudinally using multiple linear regression [5]. From a general point of view, a good overview of quantitative imaging biomarkers and their correlation with disease status and disability is given in [6].

### SUMMARY OF THE INVENTION

It is accordingly an object of the invention to provide a method and system which overcomes the above-mentioned disadvantages of the heretofore-known devices and methods of this general type and which provides for a method and a system that is capable of estimating tract-specific quantitative metrics, such as qMRI metrics, within a short examination time, in particular free of diffusion imaging, that enable a better correlation with clinical outcomes in patients, that is feasible during routine clinical examination, and the results of which can be compared between patients independently from the examination site/MRI imaging material.

With the above and other objects in view there is provided, in accordance with the invention, a computer-implemented method for mapping brain tissue damage from quantitative imaging data, the method comprising:

acquiring a quantitative map of a brain tissue parameter of the brain;

acquiring or receiving a tractography map for the brain;

superimposing a first map based on the quantitative map onto a second map based on the tractography map to form a superimposition;

extracting from the superimposition metrics reflecting a distribution of tract-specific quantitative values of the brain tissue parameter; and

displaying the metrics of the brain.

In other words, the objectives of the invention are achieved by a method and a system for imaging brain tissue damage within white matter tracts from a quantitative imaging technique, e.g. qMRI, according to the object of the independent claims. Dependent claims present further advantages of the invention.

In other words, the present invention concerns a computer-implemented method for imaging or mapping brain tissue microstructural damages from quantitative imaging data, like qMRI data, e.g. for mapping an extent of damage in white matter tracts for the brain, the method comprising:
receiving or acquiring a quantitative map, e.g. a qMRI map, of a brain tissue parameter for the brain. The quantitative map comprises notably voxels whose intensity represents a value (i.e., a measurable quantitative value) of the brain tissue parameter. For instance, the qMRI map is a T1 or a T2 map of the brain (the brain tissue parameter being then the T1 or T2 relaxation time measured in milliseconds), or is based on a combination of the latter. It can also be a map of an electrical parameter/property of the brain tissue (e.g. tissue conductivity), or a map of magnetization transfer characteristics of the brain tissue (e.g. magnetization transfer ratio (MTR), fractional pool-size, exchange rates), or any other quantifiable voxel-wise property related to the brain tissue acquired by qMRI and optionally combined with one or several other imaging techniques (e.g. computed tomography (CT) or ultrasound imaging technique) capable of quantifying a brain tissue parameter. The quantitative map, instead of being a qMRI map, might be a CT image of the brain configured for providing a map of the brain tissue parameter;
receiving or acquiring a tractography map for the brain. Preferentially, the tractography map is a brain reference image obtained from a tractography atlas (see for instance Yeh et al. [7]) or obtained from a previous diffusion-weighted (DW) MR image of the brain for which the quantitative map has been acquired, wherein “previous” means that the DW MR image has been acquired for instance in a previous examination of the brain, so that a duration of a current examination (aiming for instance to image the brain tissue damages from qMRI data) be not increased by an acquisition of diffusion-weighted MR images, and wherein the DW MR image is configured for mapping WM tractography in the brain. The tractography atlas is preferentially a public atlas built from averaged diffusion MRI data of a healthy cohort. It typically provides a whole brain tractogram, i.e., a mathematical model of brain structural connectivity composed of streamlines, wherein each streamline is configured for modeling a path followed by a fascicle of brain neuronal axons;
optionally, creating a deviation map from the quantitative map, wherein the deviation map is configured for quantifying, for each voxel of the quantitative map, a deviation of the value of the brain tissue parameter for the voxel with respect to a reference value of the brain tissue parameter for the voxel, e.g. measured or obtained when considering a healthy cohort. For this purpose, the quantitative map, e.g. the qMRI map, and a reference map of the brain tissue parameter, e.g. obtained from mapping the brain tissue parameter for the healthy cohort, might be spatially registered onto a common space, e.g. a standard space or the space of the reference map. Preferentially, the deviation map is created by determining for each voxel, the standard deviation by which its brain tissue parameter value is above or below a mean expected value estimated for that voxel in the healthy cohort;
superimposing a first (e.g. qMRI) map based on the quantitative map onto a second map based on the tractography map, after a spatial registration of the maps onto a common space, using for example non-linear registration onto the common space. The first map might be the quantitative map itself or the deviation map. The second map might be the tractography map itself, or a processed tractography map, wherein before the superimposition, the intensity value of each of the tractography map voxels has been processed for representing a number of streamlines passing through the concerned voxel;
extracting from the superimposition metrics reflecting, i.e., that are a function of, a distribution of tract-specific quantitative values of the brain tissue parameter. Such metrics are for instance, an average of the quantitative value in each voxel in the second map, preferentially, weighted by the tract density, which can be for instance, a function of the number of streamlines passing through each voxel. Instead of considering an average as metrics, other metrics can be considered such as a median, a standard deviation, a maximum, a minimum, a mode, or any other descriptive statistical metrics. The metrics can be for instance a function of a distribution of tract-specific quantitative deviations of the quantitative values with respect to a reference value, wherein the reference value is for instance a mean value, or average value, or another metric as explained above, acquired or calculated for instance from a normative atlas, wherein the set of voxels within the first map is matching a corresponding set of voxels in the second map, wherein the corresponding set of voxels has been selected by the system according to the invention, e.g. by thresholding voxel intensities in the second map; or
displaying the metrics obtained for the brain, typically by creating a map of the brain, wherein each voxel intensity value of the map represents a value of the metrics for that voxel, or reporting the metrics for different brain regions defined by the position of a set of voxels for which the metrics have been obtained.

According to the present invention, metrics are derived from quantitative MR imaging data along fiber tracks. By combining quantitative metrics from quantitative imaging, like qMRI, with spatial and functional information of the fiber tracks coming from the tractography map, the obtained metrics show increased clinical value.

With the above and other objects in view there is also provided, in accordance with the invention, a system in which the above-summarized method can be performed. The novel system is configured for mapping, preferably automatically, brain tissue microstructural damage from quantitative data, e.g. qMRI data. The system comprises:

a first interface for receiving or acquiring a quantitative map, e.g. a qMRI map, of a tissue parameter for the brain;

a second interface, which might be the same as the first interface, and which is configured for acquiring or receiving a tractography map;

a memory for storing the quantitative map and/or the tractography map;

a control unit comprising a processor, the control unit being configured for carrying out the steps of the previously described method. The control unit is thus in particular configured for using a tractography atlas and spatial registration between quantitative maps and WM tracts density maps for calculating the metrics;

a display connected to the control unit and configured for displaying the metrics obtained for the brain, e.g. via a brain map of the metrics.

The foregoing has broadly outlined the features and technical advantages of the present disclosure so that those skilled in the art may better understand the detailed description that follows. Additional features and advantages of the disclosure will be described hereinafter that form the object of the claims. Those skilled in the art will appreciate that they may readily use the concept and the specific embodiment disclosed as a basis for modifying or designing other structures for carrying out the same purposes of the present disclosure. Those skilled in the art will also realize that such equivalent constructions do not depart from the spirit and scope of the disclosure as defined by the set of claims.

The construction and method of operation of the invention, together with additional objects and advantages thereof will be best understood from the following description of specific embodiments when read in connection with the accompanying drawings.

### BRIEF DESCRIPTION OF THE FIGURES

FIG. 1 illustrates a flowchart of a method for estimating tract-specific brain tissue damages from qMRI data according to the invention; and

FIG. 2 illustrates a system for mapping brain tissue damages from qMRI data according to the invention.

### DETAILED DESCRIPTION OF THE INVENTION

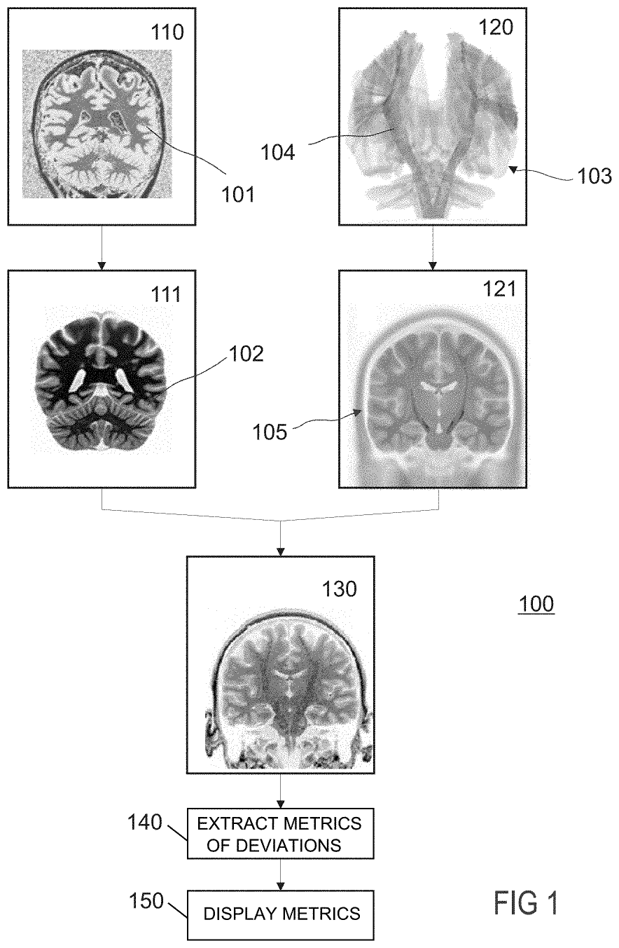

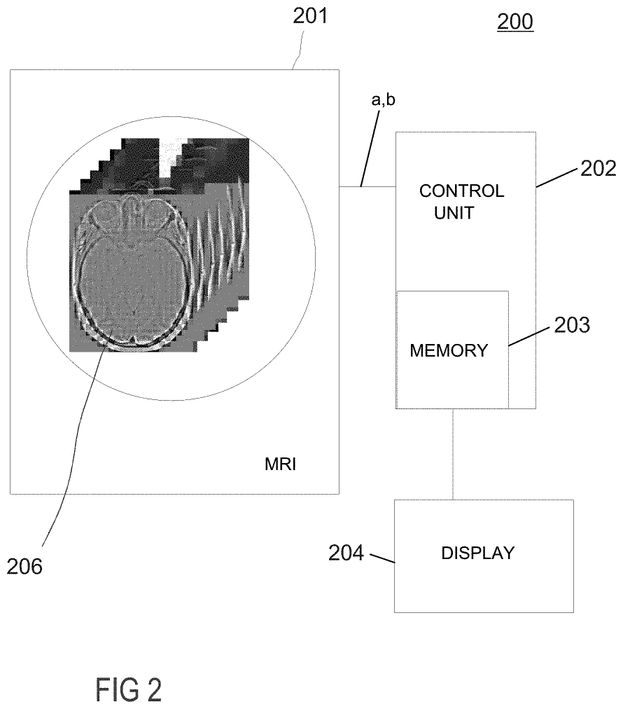

Referring now to the figures of the drawing in detail, FIGS. 1 and 2 illustrate various embodiments describing the principles of the present disclosure; the figures are for illustration only and should not be construed in any way to limit the scope of the disclosure. Those skilled in the art will understand that the principles of the present disclosure may be implemented in any suitably arranged device. The numerous innovative teachings of the present application will be described with reference to exemplary non-limiting embodiments.

FIG. 1 describes the different steps of the method 100 carried out by a preferred embodiment of the system according to the invention which is illustrated by FIG. 2.

In FIG. 2, a control unit 202 of the system 200 according to the invention is preferably connected, for instance via a first interface a, to an MRI system 201. While qMRI will be taken as illustration of the present invention, other imaging systems might be connected to the system 200 according to the invention, as long as they can provide the system 200 with a quantitative map of a brain quantitative parameter.

The MRI system 201 typically comprises different coils and respective coil controllers configured for generating magnetic fields and RF pulses in order to acquire an MRI signal from a brain 206 under investigation. The MRI signal is transmitted by a receiver coil controller to the control unit 202. The latter might be configured for reconstructing qMRI maps of the brain 206 from the MRI signal. In such a case, the control unit 202 might be configured for controlling the MRI system so that the latter performs MR imaging enabling an acquisition of qMRI maps. Alternatively or additionally, the control unit 202 might be connected to a database or any other system for acquiring or receiving, e.g. via the first interface a, qMRI maps. The control unit 202 comprises typically a memory 203 and is connected to an interface, e.g. a display 204 for displaying images reconstructed from the received MRI signal.

According to the present invention, the system 200 is configured for carrying out the following steps:

At step 110, the system 200, e.g. its control unit 202, receives or acquires one or several qMRI maps 101 of the brain 206. The qMRI maps 101 might be obtained from MRI scans of the brain according to techniques that are known in the art and that are configured for providing quantitative MRI data.

According to the present invention, a qMRI map is a brain map made of voxels, wherein the intensity value of each voxel is a measure of a brain tissue parameter obtained via a quantitative magnetic resonance imaging technique. A qMRI map according to the invention is for instance:
T1 map, measuring T1 relaxation time;
T2 map, measuring T2 relaxation time;
T2* map, measuring T2* relaxation time;
T1 ρ map, measuring T1 ρ relaxation time;
MT (magnetization transfer), measuring tissue myelination;
any diffusivity map, such as tissue fractional anisotropy;
myelin water imaging, measuring tissue myelination;
quantitative conductivity map, measuring tissue electrical conductivity;
quantitative susceptibility map, measuring tissue magnetic susceptibility;
quantitative elastography map, measuring tissue mechanical stiffness.

At step 111 and optionally, the system 200, e.g. its control unit 202, creates or computes, for the brain tissue parameter and from the acquired or received qMRI map, a deviation map 102, the latter being configured for mapping, for the brain and for each voxel, deviations of the acquired value of the brain tissue parameter with respect to a reference value mapped for that voxel in a reference map. Typically, the deviation map might be created by evaluating voxel-wise z-scores. Of course, other metrics reflecting a degree of difference between measured brain tissue parameter and a standard or reference value (e.g. mean or median value) obtained from a healthy cohort can be used. Optionally, the deviation map may be masked or thresholded to only show significant deviations.

At step 120, the system 200 receives or acquires, notably via a second interface b of the control unit 202, a brain tractography map 103. The system 200 is then preferably configured for automatically identifying, in the brain tractography map, clusters of streamlines 104 that define, each, a fiber bundle (i.e., an axonal pathway of WM tracts). Each cluster defines thus a different fiber bundle.

At step 121, the system 200, e.g. its control unit 202, is preferably configured for extracting or creating, for each streamline cluster 104, a tract density map 105 of the brain, wherein each voxel intensity value in the tract density map represents a number of streamlines of the cluster passing through that voxel. In other words, one tract density map 105 is created or extracted per tract, i.e., per streamline cluster 104. This means also that the tractography map comprises, in its whole, multiple tract density maps 105, one for each tract.

At step 130, the system 200, e.g. its control unit 202, is configured for superimposing, for each cluster, the qMRI map, or if created, the deviation map 102, and the processed tractography map, i.e., the tract density map obtained for that cluster. By superimposing, it has to be understood that the tract density map and the qMRI (or deviation) map are registered to a common space using known in the art spatial registration techniques, the common space being for instance the atlas space or the space of the brain under investigation.

At step 140, the system 200, e.g. its control unit 202, is configured for extracting, from the superimposition, metrics reflecting a distribution of tract-specific quantitative deviations. For instance, the metrics are tract-specific qMRI biomarkers extracted using aggregate statistics, configured for computing for instance a sum of voxel-wise qMRI values weighted by voxel-wise tract density values. Optionally tract-specific qMRI biomarkers might be normalized by some tract properties, like the length of the considered tract. Of course, other aggregate statistics could be used, like a (weighted) sum, mean, median or standard deviation of the values on the tract. More complex statistics could also be implemented, like an analysis of a histogram of values (e.g. peak, area under curve, etc.

At step 150, the system 200 is configured for mapping the metrics, for instance by displaying via the display 204, a map of the metrics, e.g. of the tract-specific qMRI biomarker.

Finally, the previously described invention presents the following advantages with respect to prior art techniques:
The resulting statistical tract-specific qMRI metrics are more specific to the brain function than a usual region of interest analysis;
Tract-specific statistical metrics can be estimated without requiring diffusion imaging, therefore resulting in shorter examination times, opening the way to clinical applications;
As diffusion imaging and tractography are not necessarily required, the present invention substantially improves inter-site variability induced by a use of different acquisition protocols or tractography algorithm;
Employing a tractography atlas allows using pre-existing WM tracts defined at a very fine level, which improves the precision of the resulting statistical metrics. This would be difficult to achieve with clinical DWI, mainly due to technical limitations and time constraints such as the filtering of false positive streamlines;
The interpretation of the relation between the deviation map and the tractography map is improved by extracting aggregate statistics which enables to analyze deviations for specific tracts. This is notably enabled by the spatial registration onto a common space of the tractography map with the qMRI map;
The proposed concept might be applied to various kinds of qMRI maps (e.g. T1, T2, T1rho, T2*, MTR, MWF, FA, ADC, etc.) combined or not to other quantitative information stemming from a different modality in order to compute the deviation map.

To summarize, the present invention proposes to evaluate quantitative parameters along fiber tracts, combining two pieces of information relevant for characterization of a brain disease: the notion of “microstructural tissue alteration” detected through quantitative imaging, like qMRI, and the knowledge about how the location of this tissue alteration affects a pathway, thus the brain regions connected by the pathway and hence functions which are situated in these brain regions. It has been shown that the combination of these two complementary parameters adds clinical value as the derived imaging biomarkers, i.e., the metrics, correlate better with clinical symptoms and scores.

The following is a summary list of acronyms and the corresponding structure used in the above description of the invention:

MR magnetic resonance

MRI magnetic resonance imaging

qMRI quantitative magnetic resonance imaging

MS multiple sclerosis

WM white matter

MTR magnetization transfer ratio

CT computed tomography

### LIST OF CITATIONS

[1] Barkhof F., “The clinico-radiological paradox in multiple sclerosis revisited” in Current Opinion in Neurology, 2002.
[2] Riahi, F et al. “Improved correlation between scores on the expanded disability status scale and cerebral lesion load in relapsing-remitting multiple sclerosis. Results of the application of new imaging methods.” Brain: a journal of neurology 121.7 (1998):1305-1312.
[3] Bodini, Benedetta, Marco Battaglini, et al. “T2 lesion location really matters: a 10 year follow-up study in primary progressive multiple sclerosis”. Journal of Neurology, Neurosurgery & Psychiatry 82.1 (2011):72-77.
[4] Bonnier G, Fischi-Gomez E, Roche A, et al. Personalized pathology maps to quantify diffuse and focal brain damage. Neurolmage Clin. 2019; 21:101607.
[5] Manfredonia F., et al, “Normal appearing brain T1 relaxation time predicts disability in early primary progressive multiple sclerosis”, in Archives of Neurology, 2007.
[6] deSouza, N. M., Achten, E., Alberich-Bayarri, A. et al. Validated imaging biomarkers as decision-making tools in clinical trials and routine practice: current status and recommendations from the EIBALL* subcommittee of the European Society of Radiology (ESR). Insights Imaging 10, 87 (2019).
[7] Yeh, Fang-Cheng, et al. “Population-averaged atlas of the macroscale human structural connectome and its network topology.” In Neurolmage, 2018.

1. A computer-implemented method for mapping brain tissue damage from quantitative imaging data, the method comprising:
acquiring a quantitative map of a brain tissue parameter of the brain;
acquiring or receiving a tractography map for the brain;
superimposing a first map based on the quantitative map onto a second map based on the tractography map to form a superimposition;
extracting from the superimposition metrics reflecting a distribution of tract-specific quantitative values of the brain tissue parameter; and
displaying the metrics of the brain.

2. The computer-implemented method according to claim 1, wherein the first map is a deviation map or the quantitative map itself.

3. The computer implemented method according to claim 1, wherein the second map is the tractography map itself or a processed tractography map, and the method further comprises, prior to superimposing the first map onto the second map, processing the first map for having, for each of the voxels of the first map, an intensity value of the concerned voxel quantifying a deviation of the value of the brain tissue parameter for the voxel with respect to a reference value of the brain tissue parameter for the voxel.

4. The computer-implemented method according to claim 1, wherein the extracting step comprises using aggregate statistics for extracting the metrics.

5. The computer-implemented method according to claim 4, wherein the aggregate statistics are configured for computing a sum of voxel-wise qMRI values weighted by voxel-wise tract density values.

6. A system for mapping brain tissue damage from quantitative imaging data, the system comprising:
a first interface for receiving or acquiring a quantitative map of a tissue parameter for a brain;
a second interface configured for acquiring or receiving a tractography map;
a memory for storing at least one of the quantitative map or the tractography map;
a control unit including processor, said control unit being configured for carrying out the steps of the method according to claim 1; and
a display connected to said control unit and configured for displaying the metrics obtained for the brain.
